(19) United States
(12) Patent Application Publication
(10) Pub. No.: US 2025/0207162 A1
(43) Pub. Date: Jun. 26, 2025

(54) Culture conditions for high production of antibiotic compound aureothin from Streptomyces thioluteus

(71) Applicant: KOREA ADVANCED INSTITUTE OF SCIENCE AND TECHNOLOGY, Daejeon (KR)

(72) Inventors: Byung-Kwan Cho, Daejeon (KR); Woori Kim, Daejeon (KR); Jihun Kim, Daejeon (KR); Soonkyu Hwang, Daejeon (KR)

(21) Appl. No.: 18/985,413

(22) Filed: Dec. 18, 2024

(30) Foreign Application Priority Data
Dec. 21, 2023 (KR) 10-2023-0188637

(51) Int. Cl. C12P 17/16; C12N 1/20

(52) U.S. Cl. CPC C12P 17/162; C12N 1/20

(57) ABSTRACT

The present invention relates to culture conditions for high production of the antibiotic aureothin from Streptomyces thioluteus.

### CROSS-REFERENCE TO RELATED APPLICATIONS

This application claims priority to Republic of Korea Patent Application Serial No. 10-2023-0188637, filed on Dec. 21, 2023, the entire contents of which are incorporated herein by reference.

### TECHNICAL FIELD OF THE INVENTION

The present invention relates to culture conditions for the high production of the antibiotic aureothin from Streptomyces thioluteus.

### BACKGROUND OF THE INVENTION

Aureothin is a useful substance that exhibits antibiotic, antitumor, antifungal, and insecticidal effects. Among previous research results for mass production thereof, there is a research result showing that the heterologous expression of the aureothin biosynthetic gene cluster (BGC) in Streptomyces avermitilis SUKA22 produced 116 mg/L of aureothin (Komatsu M, Komatsu K, Koiwai H, Yamada Y, Kozone I, Izumikawa M, Hashimoto J, Takagi M, Omura S, Shin-ya K, Cane D E, Ikeda H. Engineered Streptomyces avermitilis host for heterologous expression of biosynthetic gene cluster for secondary metabolites. ACS Synth Biol. 2013 Jul. 19; 2 (7): 384-96. doi: 10.1021/sb3001003. Epub 2013 Jan. 17. PMID: 23654282; PMCID: PMC3932656), and this was the highest concentration of production of aureothin reported so far.

The present inventors have made extensive efforts to achieve a high production of aureothin more efficiently and as a result, developed a culture method capable of attaining a production yield of up to 376.55 mg/L, and thus the present inventors have completed the present invention.

### SUMMARY OF THE INVENTION

### Technical Problem

An aspect of the present invention is to provide a method for producing an antibiotic, the method including: culturing Streptomyces thioluteus in a medium containing a specific composition; and recovering an antibiotic from the cultured microorganism or medium.

Another aspect of the present invention is to provide a culture method for increasing the production of an antibiotic from Streptomyces thioluteus, the method including culturing Streptomyces thioluteus in a medium containing a specific composition.

### Technical Solution

In accordance with one aspect of the present inventiona method for producing an antibiotic is provided, which includes: culturing Streptomyces thioluteus in a medium; and recovering an antibiotic from the cultured microorganism or medium.

In an embodiment, the method for producing an antibiotic of the present invention may include culturing Streptomyces thioluteus in a medium containing any one composition of (1) to (4) below:
(1) sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L;
(2) yeast extract 4 g/L, malt extract 10 g/L, and maltose 4 g/L;
(3) yeast extract 4 g/L, malt extract 10 g/L, and glucose 4 g/L; and
(4) soluble starch 10 g/L, glucose 20 g/L, soy peptone 25 g/L, beef extract 1 g/L, yeast extract 4 g/L, sodium chloride 2 g/L, and calcium carbonate 2 g/L.

In another embodiment, the antibiotic may be aureothin.

In the method according to any one of the above-described embodiments, the medium may contain any one composition of (1) to (3) below:
(1) sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L;
(2) yeast extract 4 g/L, malt extract 10 g/L, and maltose 4 g/L; and
(3) yeast extract 4 g/L, malt extract 10 g/L, and glucose 4 g/L.

In the method according to any one of the above-described embodiments, the medium may contain sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L.

In the method according to any one of the above-described embodiments, the method may include pre-culturing the strain and main culturing the strain.

In the method according to any one of the above-described embodiments, the pre-culturing may be performed for 12 hours to 36 hours.

In the method according to any one of the above-described embodiments, the main culturing may be performed for 144 hours to 192 hours.

In the method according to any one of the above-described embodiments, the method may further include isolating and/or purifying the recovered antibiotic.

In another aspect of the present invention, there is provided a culture method for increasing the production of an antibiotic from Streptomyces thioluteus, the method including culturing Streptomyces thioluteus in a medium containing any one composition of (1) to (4) below.

In an embodiment, the culture method for Streptomyces thioluteus of the present invention may increase the production of an antibiotic by culturing Streptomyces thioluteus in a medium containing any one composition of (1) to (4) below:
(1) sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L;
(2) yeast extract 4 g/L, malt extract 10 g/L, and maltose 4 g/L;
(3) yeast extract 4 g/L, malt extract 10 g/L, and glucose 4 g/L; and
(4) soluble starch 10 g/L, glucose 20 g/L, soy peptone 25 g/L, beef extract 1 g/L, yeast extract 4 g/L, sodium chloride 2 g/L, and calcium carbonate 2 g/L.

In another embodiment, the culture method for Streptomyces thioluteus of the present invention may increase the production of aureothin.

In the culture method according to any one of the above-described embodiments, the medium may contain any one composition of (1) to (3) below:
(1) sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L;
(2) yeast extract 4 g/L, malt extract 10 g/L, and maltose 4 g/L; and
(3) yeast extract 4 g/L, malt extract 10 g/L, and glucose 4 g/L.

In the culture method according to any one of the above-described embodiments, the medium may contain sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L.

In the culture method according to any one of the above-described embodiments, the culture method may increase aureothin production by at least five times compared to when the same strain is cultured using, as a culture medium, a medium containing yeast extract 3 g/L, malt extract 3 g/L, peptone 5 g/L, and glucose 4 g/L for the same time.

In another aspect of the present invention, there is provided a culture medium for Streptomyces thioluteus for antibiotic production.

In an embodiment, the culture medium for Streptomyces thioluteus for antibiotic production may contain any one composition of (1) to (4) below:
(1) sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L;
(2) yeast extract 4 g/L, malt extract 10 g/L, and maltose 4 g/L;
(3) yeast extract 4 g/L, malt extract 10 g/L, and glucose 4 g/L; and
(4) soluble starch 10 g/L, glucose 20 g/L, soy peptone 25 g/L, beef extract 1 g/L, yeast extract 4 g/L, sodium chloride 2 g/L, and calcium carbonate 2 g/L.

In another embodiment, in the culture medium for Streptomyces thioluteus for antibiotic production, the produced antibiotic is aureothin.

The present invention will be specifically described as follows. Each description and exemplary embodiment disclosed herein may also be applied to other descriptions and exemplary embodiments. That is, all combinations of various elements disclosed herein fall within the scope of the present invention. Furthermore, the scope of the present invention is not limited by the specific description below.

Furthermore, a person skilled in the art will recognize or be able to ascertain many equivalents to the specific embodiments of the present invention described herein, by using no more than routine experimentation. Furthermore, these equivalents are intended to be included in the present invention.

Furthermore, throughout the overall specification, many papers and patent documents are referenced and their citations are provided. The disclosures of cited papers and patent documents are entirely incorporated by reference herein, and the level of the technical field within which the present invention falls and details of the present invention are explained more clearly.

As used herein, the term “medium” refers to a substance in which nutrients required for culturing microorganisms are mixed as main components. The medium serves to supply nutrients, growth factors, and others, including water that is essential for survival and growth.

In the present invention, the use of a medium having a specific composition maximized the production of an antibiotic from a cultured strain.

The medium containing (1) sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L in the present invention may be named as R5(−) medium.

The medium containing (2) yeast extract 4 g/L, malt extract 10 g/L, and maltose 4 g/L in the present invention may be named as MYM medium.

The medium containing (3) yeast extract 4 g/L, malt extract 10 g/L, and maltose 4 g/L in the present invention may be named as ISP2 medium.

The medium containing (4) soluble starch 10 g/L, glucose 20 g/L, soy peptone 25 g/L, beef extract 1 g/L, yeast extract 4 g/L, sodium chloride 2 g/L, and calcium carbonate 2 g/L in the present invention may be named as GSS medium, and MB-G0666 GSS Broth, a commercially available product, may be used.

In the composition of the medium of the present invention, distilled water may be further contained in addition to components not listed.

The R5(−) medium of the present invention may further contain trace elements in addition to the above-described components. For example, the trace elements may be added after sterilization. For instance, the trace elements may further include a component selected from ZnCl₂, FeCl₃, CuCl₂, MnCl₂, Na₂B₄O₇, (NH₄)₆Mo₇O2₄, and hydrates thereof. For instance, the medium may contain ZnCl₂, FeCl₃·6H₂O, CuCl₂·2H₂O, MnCl₂·4H₂O, Na₂B₄O₇·10H₂O, and (NH₄)₆Mo₇O₂₄·4H₂O.

In an embodiment of the present invention, the medium of the present invention was prepared by autoclaving the R5(−) medium and then adding a trace element solution containing ZnCl₂ 0.08 mg, FeCl₃·6H₂O 0.4 mg, CuCl₂·2H₂O 0.02 mg, MnCl₂·4H₂O 0.02 mg, Na₂B₄O₇·10H₂O 0.02 mg, and (NH₄)₆Mo₇O₂₄·4H₂O 0.02 mg and NaOH for pH adjustment thereto.

The medium of the present invention may be autoclaved. Specifically, the medium may be sterilized by autoclaving, but is not limited thereto.

In the present invention, a microorganism of the genus Streptomyces may be cultured while known conventional culture conditions, for example, pH and temperature, are adjusted. The temperature of the culture may be 25 to 40° C., and specifically 28 to 37° C., but is not limited thereto. The culture period may continue until the useful substance is obtained at the desired production level.

The Streptomyces thioluteus of the present invention may be a wild type strain or a genetically engineered recombinant strain. Particularly, the recombinant strain may involve genetic manipulation to increase the production of a desired antibiotic. For example, the strain of the present invention may have antibiotic production ability, for example, aureothin production ability.

Herein, “aureothin” is a compound, of which the IUPAC name is 2-methoxy-3,5-dimethyl-6-[(2R,4Z)-4-[(E)-2-methyl-3-(4-nitrophenyl) prop-2-enylidene]oxolan-2-yl]pyran-4-one, with a structure of Chemical Formula 1 below:

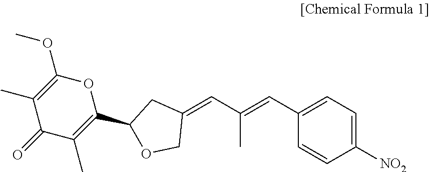 [SMILES: COc1oc([C@H]2C/C(=C/C(C)=C/c3ccc([N+](=O)[O-])cc3)CO2)c(C)c(=O)c1C]

Aureothin is a substance known to have an antifungal and antibacterial effects and to inhibit oxidoreductase.

In an embodiment of the present invention, after researching and developing a culture method capable of maximizing the aureothin production ability, it was identified that the culture of Streptomyces thioluteus in a medium having a specific composition significantly increased the aureothin production ability. Specifically, in a medium containing a composition of (1) sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L, (2) yeast extract 4 g/L, malt extract 10 g/L, and maltose 4 g/L, (3) yeast extract 4 g/L, malt extract 10 g/L, and glucose 4 g/L, or (4) soluble starch 10 g/L, glucose 20 g/L, soy peptone 25 g/L, beef extract 1 g/L, yeast extract 4 g/L, sodium chloride 2 g/L, and calcium carbonate 2 g/L, Streptomyces thioluteus was pre-cultured for 1-2 days and then main cultured for 7 days, thereby confirming an aureothin production of up to 376.55 mg/L.

### Advantageous Effects

A large amount of aureothin can be efficiently produced through the method of the present invention.

### DETAILED DESCRIPTION OF THE INVENTION

For a more complete understanding of the features and advantages of the present invention, reference is now made to the detailed description of the invention along with the accompanying figures and in which:

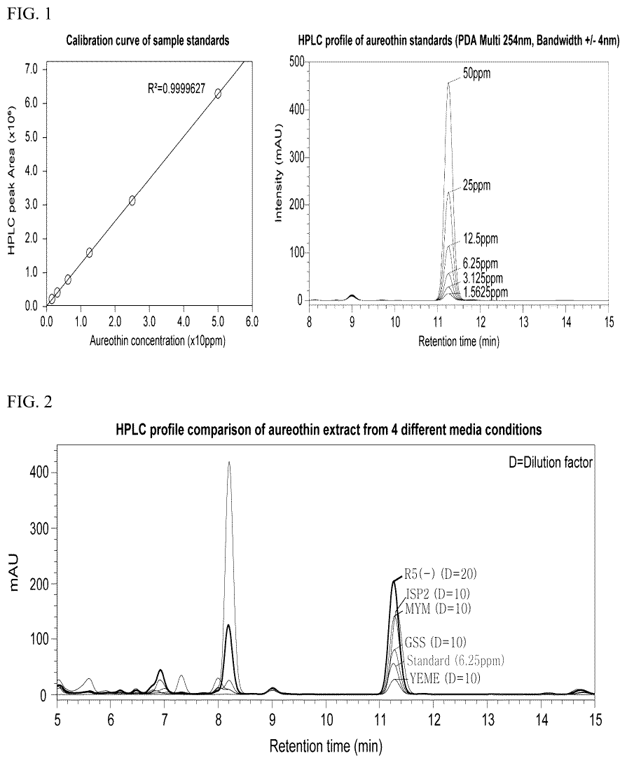

FIG. 1 shows the calibration curve results of aureothin measurement.

FIG. 2 shows the results of aureothin production measurement under five different culture conditions (D=10 indicates a 10-fold dilution sample, and D=20 indicates a 20-fold dilution sample).

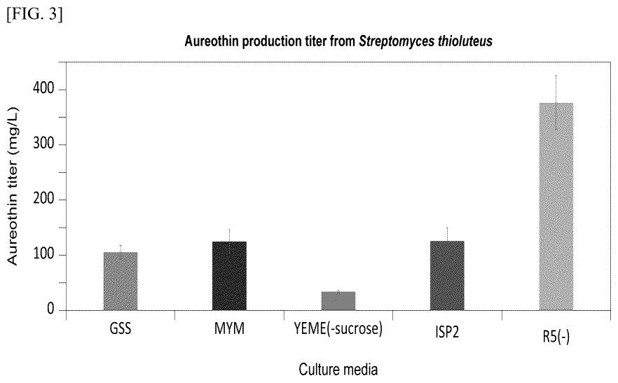

FIG. 3 shows the results of aureothin production under different culture conditions.

### DETAILED DESCRIPTION OF PREFERRED EMBODIMENTS

Hereinafter, the present invention will be described in detail with reference to examples and experimental examples. However, these examples and experimental examples are given for specifically illustrating the present disclosure, and the scope of the present disclosure is not limited thereto.

### Example 1: Materials and Methods

S. thioluteus DSM 40027 (wild type) was used as the strain in the present invention.

The cell culture method used in the present invention was as follows.
(1) A 250-mL baffled flask, in which 8 g of glass beads were placed, was autoclaved.
(2) Into the prepared 250-mL baffled flask, 50 mL of each medium, prepared by autoclaving, was dispensed.
(3) Pre-culturing step: A 100-μL stock of S. thioluteus DSM 40027 strain was inoculated into the medium prepared in (2), and then cultured in a shaking incubator at 200 rpm for 1 to 2 days at 30° C.
(4) Each medium (50 mL) was prepared in a 250-mL baffled flask containing glass beads, prepared through (1) and (2) (triplicate).
(5) Main culturing step: A portion of the cell medium cultured in the pre-culturing step of (3) was sampled, and the O.D. value (at 600 nm) thereof was measured using a spectrophotometer.

The cell culture was added to the flask prepared in (4) to an initial O.D. value of 0.05, and then cultured in a shaking incubator at 200 rpm for 7 days at 30° C.

The cell culture used in the present invention was analyzed as follows.
(1) After culturing for 7 days, a portion of the culture was sampled, and then the O.D. value at 600 nm was measured using a spectrophotometer to investigate cell growth.
(2) After 1 mL of the culture was sampled, 1 mL of ethyl acetate was added, mixed by vortexing, and rotated in a multi-mixer for 10 minutes. After the resultant culture was centrifuged at 16,000 g for 10 minutes, 800 μL of the ethyl acetate layer was recovered and transferred to a new 2-mL EP tube. To the culture remaining after the transfer of the ethyl acetate layer, 800 μL of ethyl acetate was further added, mixed by vortexing, rotated in a multi-mixer for 10 minutes, and then centrifuged using a centrifuge at 16,000 g for 10 minutes. Then, 800 μL of the ethyl acetate layer was transferred to a 2-mL EP tube to obtain a total of 1.8 mL of an ethyl acetate extract.
(3) Ethyl acetate was completely evaporated in a fume hood.
(4) The resultant product was mixed, by vortexing, with 800 μL of methanol added, and diluted 1/10 with methanol.
(5) The resultant product was filtered with a 0.22-μm Minisart RC syringe filter and subjected to HPLC analysis, thereby measuring the concentration of produced aureothin. After PDA measurement, the results at 254 nm wavelength were examined.

The culture media used in the present invention were as follows. The same medium was used for main culturing and pre-culturing. For example, if the medium for pre-culturing was GSS, the medium for main culturing was also GSS.

TABLE 1
GSS (MB-G0666 GSS Broth Product)

| Composition | Weight/Volume | |
|---|---|---|
| Soluble Starch | 10 | g |
| Glucose | 20 | g |
| Soy Peptone | 25 | g |
| Beef Extract | 1 | g |
| Yeast Extract | 4 | g |
| Sodium Chloride | 2 | g |
| Calcium Carbonate | 2 | g |
| Distilled Water | 1 | L |

Autoclave

TABLE 2
MYM

| Composition | Weight/Volume | |
|---|---|---|
| Yeast extract | 4 | g |
| Malt extract | 10 | g |
| Maltose | 4 | g |
| Distilled Water | 1 | L |

Autoclave

TABLE 3
YEME-sucrose

| Composition | Weight | |
|---|---|---|
| Yeast extract | 3 | g |
| Malt extract | 3 | g |
| Peptone | 5 | g |
| Glucose | 10 | g |
| Distilled Water | 1 | L |

Autoclave

TABLE 4
ISP2

| Composition | Weight | |
|---|---|---|
| Yeast extract | 4 | g |
| Malt extract | 10 | g |
| Glucose | 4 | g |
| Distilled Water | 1 | L |

Autoclave

TABLE 5
R5(—)

| Composition | Weight/Volume | |
|---|---|---|
| Sucrose | 103 | g |
| K₂SO₄ | 0.25 | g |
| MgCl₂•6H₂O | 10.12 | g |
| Glucose | 10 | g |
| Casamino acids | 0.1 | g |
| Yeast extract | 5 | g |
| TES buffer | 5.73 | g |
| Distilled Water | 1 | L |

Autoclave

| After autoclave | Volume(mL) |
|---|---|
| NaOH (1N) | 7 |
| Trace element solution | 0.2 |

TABLE 6
Composition of trace element solution in R5(—)

| Composition | Weight/Volume | |
|---|---|---|
| ZnCl₂ | 0.08 | g |
| FeCl₃•6H₂O | 0.4 | g |
| CuCl₂•2H₂O | 0.02 | g |
| MnCl₂•4H₂O | 0.02 | g |
| Na₂B₄O₇•10H₂O | 0.02 | g |
| (NH₄)₆Mo₇O₂₄•4H₂O | 0.02 | g |
| Distilled Water | 200 | mL |

Autoclave

The analytical conditions used for quantifying aureothin produced in the present invention were as follows.
(1) UV-vis spectrophotometer: Absorbance at 600 nm
(2) HPLC
Flow rate: 0.6 mL/min
Column temperature: 40° C.
Injection volume: 20 μL
Solvent A: MilliQ water
Solvent B: 100% HPLC grade Acetonitrile
Condition: 70% solvent B (isocratic)
Time: 50 min
Detector: PDA
(3) Sample preparation

The microbial culture was subjected to extraction with twice the volume of ethyl acetate, and the ethyl acetate layer was evaporated. Thereafter, the supernatant obtained by dissolution in methanol with an equal volume and centrifugation was filtered with a 0.22-μm RC syringe filter and then diluted 1:10 with methanol.

### Example 2: Experimental Results

In the present invention, the calibration curve generation results during aureothin production measurement are shown in FIG. 1. Aureothin samples with 50, 25, 12.5, 6.25, 3.125, and 1.5625 ppm were measured as standard samples, and the R2 value of the calibration curve was 0.9999627.

The measurement results of aureothin production of Streptomyces thioluteus under conditions using five types of media in Example 1 are shown in FIG. 2.

The peaks at a retention time of around 11 min were identified as aureothin when compared to the standard samples, and the highest production was observed under R5(−) culture medium conditions. The aureothin production titer for each type of culture condition considering the dilution factors is shown as the average of measurement values through three repeated experiments in FIG. 3.

The aureothin productions under five medium conditions were 105.6 mg/L (GSS), 124.52 mg/L (MYM), 34.04 mg/L (YEME-sucrose), 125.7 mg/L (ISP2), and 376.55 mg/L (R5(−)). Of these, MYM, ISP2, and R5(−) medium conditions showed titers higher than the 116 mg/L produced by heterologous expression in Streptomyces avermitilis SUKA22 according to the previous document. Especially, the use of R5(−) medium showed a significant increase in aureothin production compared with the other culture conditions, more especially, showing an aureothin production at least ten times higher compared with that under the YEME-sucrose conditions.

While the present invention has been described with reference to the particular illustrative embodiments, a person skilled in the art to which the present invention pertains can understand that the present invention may be embodied in other specific forms without departing from the technical spirit or essential characteristics thereof. Therefore, the embodiments described above should be construed as being exemplified and not limiting the present invention. The scope of the invention should be construed according to the meaning and scope of the appended claims rather than the detailed description and all changes or variations derived from equivalent concepts fall within the scope of the present invention.

What is claimed is:

1. A method for producing an antibiotic, the method comprising: culturing Streptomyces thioluteus in a medium containing a specific composition; and recovering an antibiotic from the cultured microorganism or medium,
wherein the medium contains any one composition of (1) to (4) below:
(1) sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L;
(2) yeast extract 4 g/L, malt extract 10 g/L, and maltose 4 g/L;
(3) yeast extract 4 g/L, malt extract 10 g/L, and glucose 4 g/L; and
(4) soluble starch 10 g/L, glucose 20 g/L, soy peptone 25 g/L, beef extract 1 g/L, yeast extract 4 g/L, sodium chloride 2 g/L, and calcium carbonate 2 g/L.

2. The method of claim 1, wherein the antibiotic is aureothin.

3. The method of claim 1, wherein the medium contains any one composition of (1) to (3) below:
(1) sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L;
(2) yeast extract 4 g/L, malt extract 10 g/L, and maltose 4 g/L; and
(3) yeast extract 4 g/L, malt extract 10 g/L, and glucose 4 g.

4. The method of claim 1, wherein the medium contains sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L.

5. A culture method for increasing the production of an antibiotic from Streptomyces thioluteus, the method comprising culturing Streptomyces thioluteus in a medium containing any one composition of (1) to (4) below:
(1) sucrose 103 g/L, K₂SO₄ 0.25 g/L, MgCl₂·6H₂O 10.12 g/L, glucose 10 g/L, casamino acids 0.1 g/L, yeast extract 5 g/L, and N-[tris(hydroxymethyl)methyl]-2-aminoethanesulfonic acid (TES) buffer 5.73 g/L;
(2) yeast extract 4 g/L, malt extract 10 g/L, and maltose 4 g/L;
(3) yeast extract 4 g/L, malt extract 10 g/L, and glucose 4 g/L; and
(4) soluble starch 10 g/L, glucose 20 g/L, soy peptone 25 g/L, beef extract 1 g/L, yeast extract 4 g/L, sodium chloride 2 g/L, and calcium carbonate 2 g/L.
